(19) United States
(12) Patent Application Publication
(10) Pub. No.: US 2017/0086734 A1
(43) Pub. Date: Mar. 30, 2017

(54) SYSTEMS AND METHODS FOR TREATING HYPOTHERMIA

(71) Applicant: Elwha LLC, Bellevue, WA (US)

(72) Inventors: Roderick A. Hyde, Redmond, WA (US); Muriel Y. Ishikawa, Livermore, CA (US); Jordin T. Kare, San Jose, CA (US); Eric C. Leuthardt, St. Louis, MO (US); Dennis J. Rivet, Chesapeake, VA (US); Lowell L. Wood, JR., Bellevue, WA (US)

(21) Appl. No.: 14/864,195

(22) Filed: Sep. 24, 2015

(51) Int. Cl. A61B 5/00; A61B 5/01; A61B 46/00; A61N 5/02; A61N 2/00; A61F 7/00; A61B 5/055

(52) U.S. Cl. CPC A61B 5/4836; A61F 7/00; A61B 5/01; A61B 5/055; A61B 5/6846; A61N 5/025; A61N 2/004; A61B 46/40

(57) ABSTRACT

Embodiments disclosed herein are directed to systems and methods for treating hypothermia in a subject is disclosed. In an embodiment, a method includes determining or measuring a temperature of a target region of the subject. The method also includes responsive to determining or measuring the temperature, directing electromagnetic energy at an external surface of the target region of the subject effective to heat the target region to a temperature of less than an ablation temperature of the target region.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

Embodiments disclosed herein are directed to systems and methods for treating hypothermia. In an embodiment, a hypothermia treatment system includes a support structure, an electromagnetic energy source, at least one temperature sensor, and a control system. The support structure is configured to support a subject having a target region thereon. The electromagnetic energy source is configured to output electromagnetic energy toward the target region of the subject to selectively heat the target region. The electromagnetic energy source is located external to the subject. The at least one temperature sensor is configured to determine or measure a temperature of the target region of the subject. The control system is operably coupled to the electromagnetic energy source and the at least one temperature sensor. The control system is configured to control at least one operational parameter of the electromagnetic energy output by the electromagnetic energy source responsive to the at least one temperature sensor determining or measuring the temperature of the target region so that the temperature of the target region is maintained below a tissue damaging temperature of the target region.

In an embodiment, a method for treating hypothermia in a subject is disclosed. A temperature of a target region of the subject, such as a subsurface target region, is determined or measured. Responsive to determining or measuring the temperature, electromagnetic energy is directed at an external surface of a target region of the subject effective to heat the target region to a temperature of less than an ablation temperature of the target region.

Features from any of the disclosed embodiments can be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1A is a schematic diagram of an embodiment of a hypothermia treatment system.

FIG. 1B is a schematic diagram of an embodiment of a hypothermia treatment system including a medical imaging system.

FIG. 1C is a schematic diagram of an embodiment of a hypothermia treatment system including a thermography system.

FIG. 1D is a schematic diagram of an embodiment of a hypothermia treatment system including a reflective material.

FIG. 1E is a schematic diagram of an embodiment of a hypothermia treatment system including an energy absorptive material.

FIG. 2A is a schematic diagram of an embodiment of a hypothermia treatment system including an array of sensors.

FIG. 2B is a schematic diagram of an embodiment of a hypothermia treatment system including an array of electromagnetic energy sources.

FIG. 3 is a schematic diagram of an embodiment of a hypothermia treatment system including a supply of electromagnetic energy absorption agents.

FIG. 4A is a schematic diagram of an embodiment of a hypothermia treatment system including one or more components integrated into a support structure.

FIG. 4B is a schematic diagram of an embodiment of a hypothermia treatment system including at least one temperature sensor deployed internally within a patient.

FIG. 5 is a schematic diagram of an embodiment of a hypothermia treatment system including a movable electromagnetic energy source.

FIG. 6 is a schematic diagram of an embodiment of a hypothermia treatment system including a support structure including a chair.

### DETAILED DESCRIPTION

Embodiments disclosed herein are directed to systems and methods for treating hypothermia. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be strictly limiting. Other embodiments may be utilized, and other changes may be made, without departing from the spirit or scope of the subject matter presented herein.

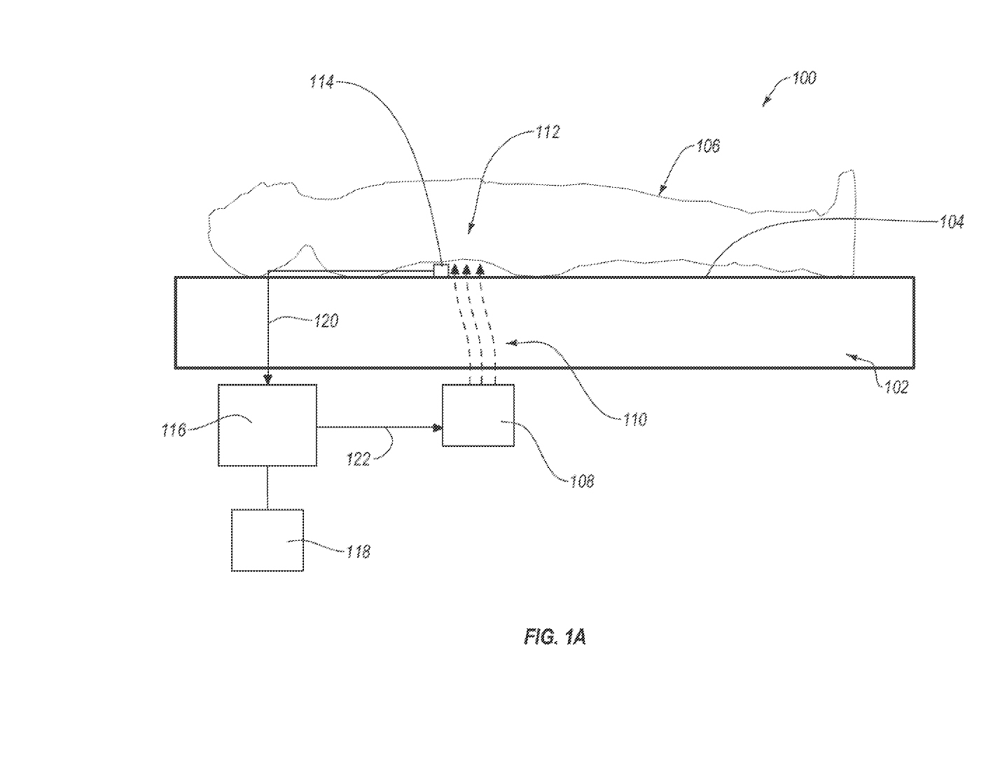

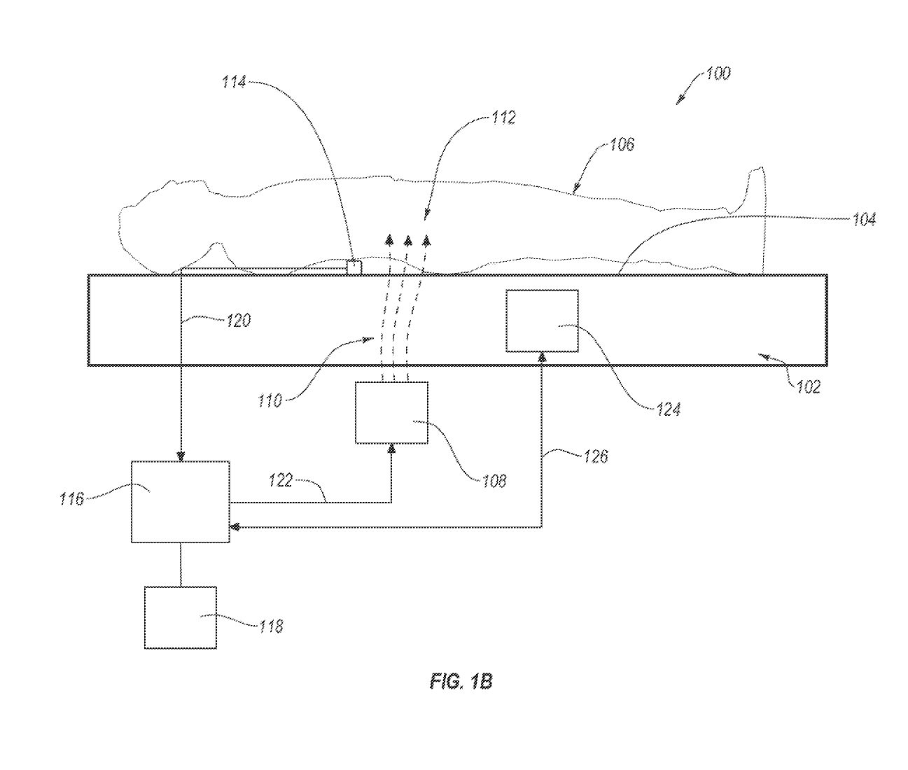

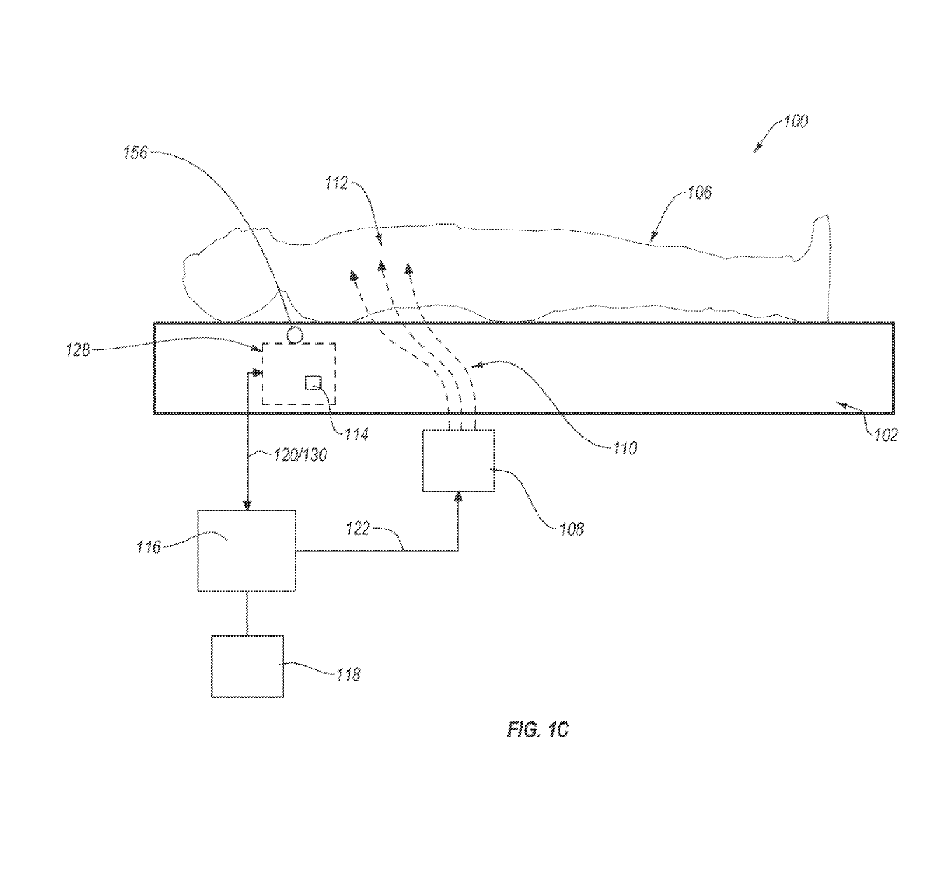

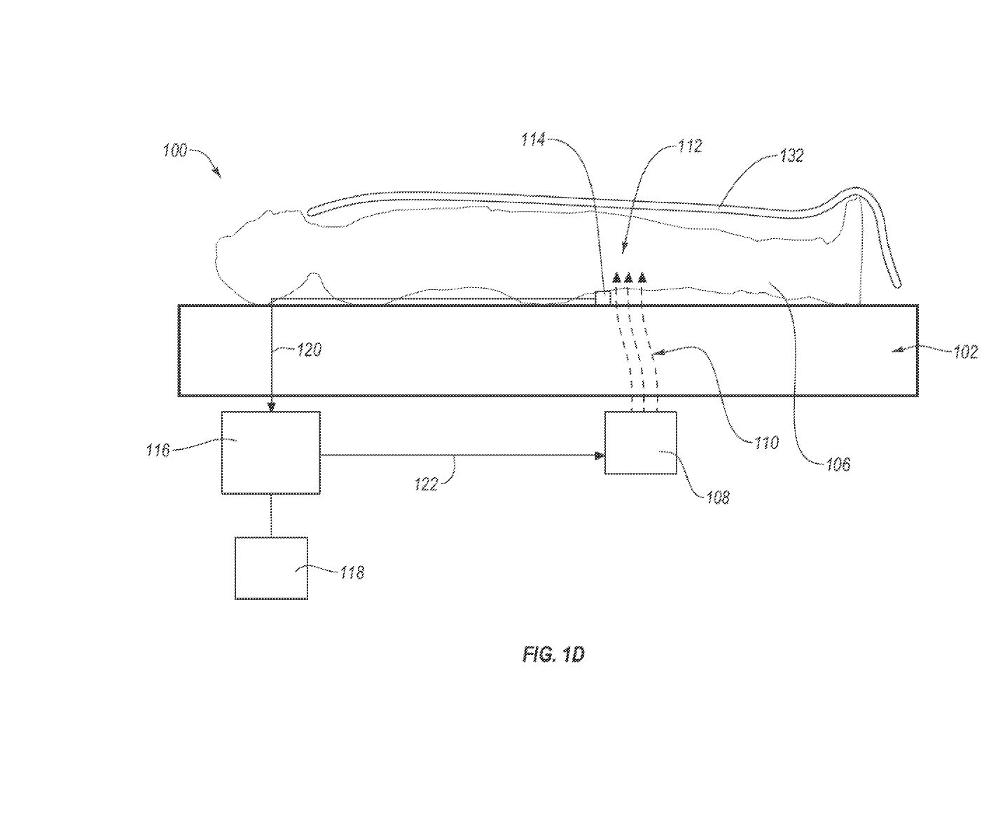

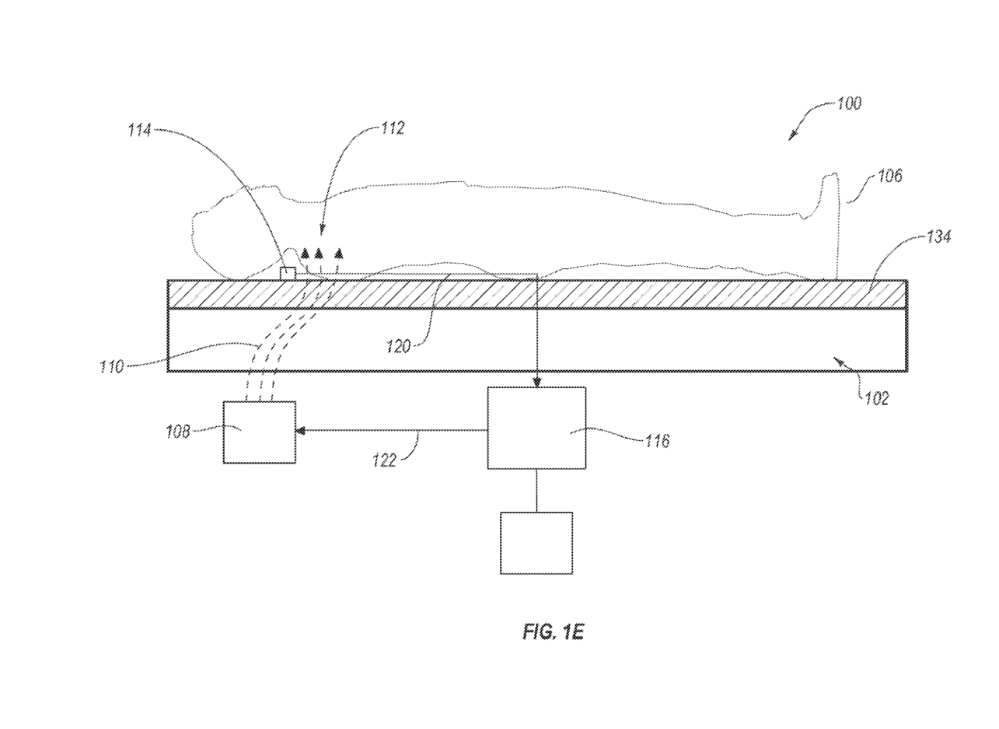

FIG. 1A is a schematic diagram of a hypothermia treatment system 100, according to an embodiment. The hypothermia treatment system 100 includes a support structure 102 configured to support the subject 106 thereon. For example, the support structure 102 can include a support surface 104 that supports the subject 106. In an embodiment, the support surface 104 is substantially rigid. In an embodiment, the support surface 104 can be flexible. In an embodiment, the support surface 104 includes a substantially rigid portion and a substantially flexible portion. In an embodiment, the support surface 104 can include one or more cushioning materials. The support structure 102 can exhibit a variety of different configurations selected for a particular application. For example, the support structure 102 can include a chair, a bed, a surgical table, a stretcher, a gurney, a platform, a couch, a sleeping bag, or a hypothermia wrap. The support structure 102 can include a patient support structure or a subject support structure.

In an embodiment, the support structure 102 can include any suitable conventional operating table. For example, the support structure 102 can include, but is not limited to, the Elite 6300 General Purpose Table, commercially available from Skytron, Grand Rapids, Mich.; The Alphamaquet 1150, commercially available from MAQUET Holding GmbH & Co. KG, Rastatt, Germany; the DRE Versailles P100 Powered Mobile Surgery Table, commercially available from DRE, Inc., Louisville, Ky. Of course, other individually adapted operating tables can be employed for the support structure 102.

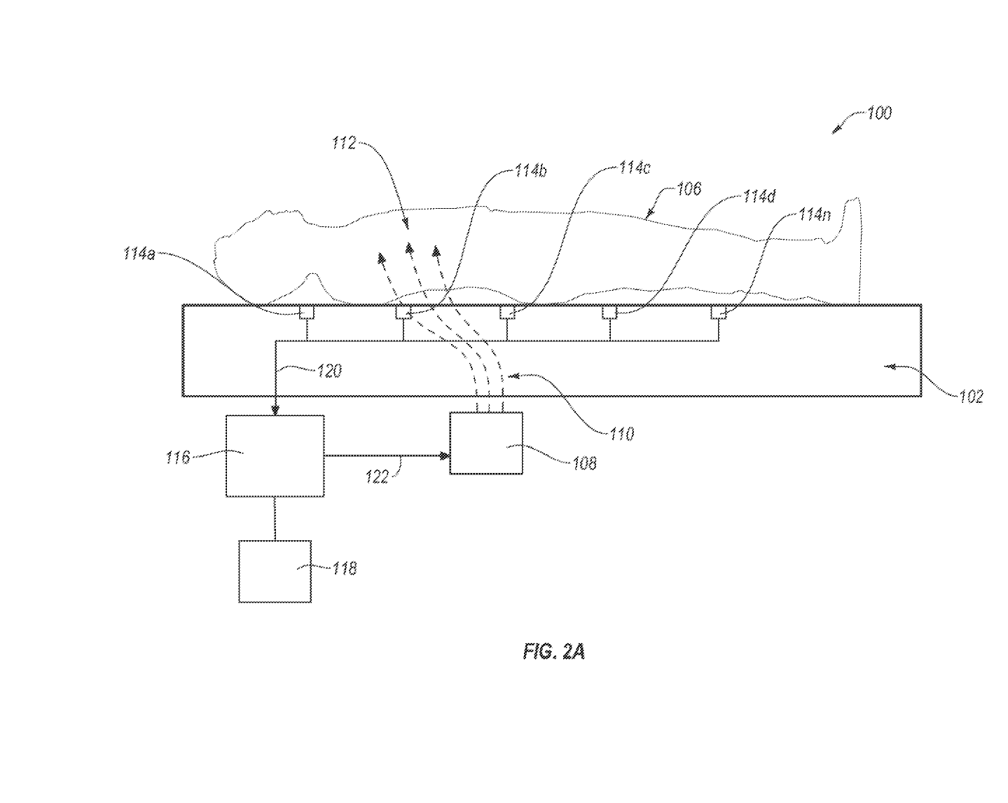

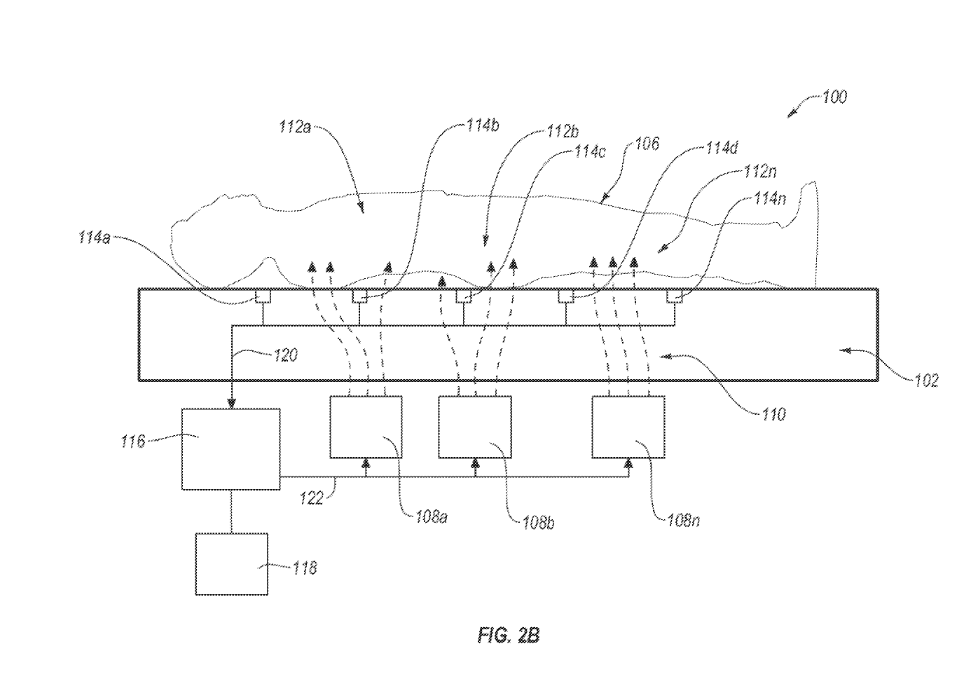

The hypothermia treatment system 100 further includes an electromagnetic energy source 108. In the illustrated embodiment, the electromagnetic energy source 108 is positioned under the support structure 102. However, in an embodiment, the electromagnetic energy source 108 is incorporated in the support structure 102. In an embodiment, the electromagnetic energy source 108 is positioned over the support structure 102. In an embodiment, the electromagnetic energy source 108 is positioned around at least a portion of the subject 106. For example, the electromagnetic energy source 108 can be wrapped around at least a portion of a leg of the subject 106. In an embodiment, the electromagnetic energy source 108 is wrapped around the back of the subject 106. In an embodiment, the hypothermia treatment system 100 can include a plurality of electromagnetic energy sources 108 (shown in FIG. 2B).

In an embodiment, the electromagnetic energy source 108 is configured to selectively output electromagnetic energy 110 into the subject 106 in order to treat hypothermia. In an embodiment, the electromagnetic energy source 108 is configured to selectively output electromagnetic energy 110 into the subject 106 in order to prevent hypothermia. For example, the electromagnetic energy source 108 can be configured to selectively output electromagnetic energy 110 into the subject 106 to provide uniform heating over the target region 112 (e.g., the volume of the subject 106) during surgery to prevent patient hypothermia. Hypothermia is characterized by a lowering of core body temperature below physiological normal limits, which is typically less than 35° C. for a human and results when a subject's body heat loss exceeds body heat production. Hypothermia can be classified as accidental or intentional, primary or secondary, and by the degree of hypothermia. Accidental hypothermia can result from unanticipated exposure to cold and wet conditions. Intentional hypothermia is an induced state generally directed at neuroprotection after an at-risk situation. Primary hypothermia is due to environmental exposure, with no underlying medical condition causing disruption of temperature regulation. Secondary hypothermia is low body temperature resulting from a medical illness. Hypothermia can be life-threatening, impairing neurological, cardiovascular, respiratory, and gastrointestinal systems. In an embodiment, the subject 106 is a patient in surgery. In an embodiment, the subject 106 is a patient being treated for primary hypothermia. In an embodiment, the subject 106 is a patient being treated for secondary hypothermia. In an embodiment, the subject 106 is being treated for accidental hypothermia. In an embodiment, the subject 106 is a patient in an induced state of hypothermia. In an embodiment, the subject is a human. However, in an embodiment, the subject 106 can include any warm-blooded animal susceptible to hypothermia, including, but not limited to, a horse, a canine, a feline, primates, or cattle.

In an embodiment, the electromagnetic energy source 108 can selectively output electromagnetic energy 110 toward a target region 112 of the subject 106 to heat the target region 112. In an embodiment, the target region 112 can include a surface region including, but not limited to, the neck, the armpits, the head, the groin, palms of the hands, the chest, the abdomen, or the back. In an embodiment, the target region 112 can include a subsurface region including, but not limited to, internal body organs (e.g., the brain, the heart, the lungs, the kidneys), the thorax, the gastrointestinal tract, the vertebral arteries, the common carotid arteries, the internal carotid artery, the external carotid artery, the axillary artery, the brachial artery, the ulnar artery, the radial artery, the femoral artery, the popliteal artery, the tibial arteries, or the dorsal pedal artery. In an embodiment, the target region 112 can include surface regions and subsurface regions. In an embodiment, the target region 112 can include one or more locations.

In an embodiment, the electromagnetic energy source 108 can include an array of electromagnetic energy sources to substantially, uniformly heat the target region 112. In an embodiment, the electromagnetic energy source 108 can include a scanning system to substantially, uniformly heat the target region 112. In an embodiment, the electromagnetic energy source 108 can include an antenna system that continuously shifts the electromagnetic energy 110 over the target region 112 to substantially uniformly heat the target region 112. For example, the electromagnetic energy source 108 can include an array of antennas configured to transmit the electromagnetic energy 110 toward the target region 112. By controlling one or more of the antennas, the control system 116 can direct the electromagnetic energy source 110 to substantially uniformly heat the target region 112.

In an embodiment, the heat produced by the electromagnetic energy 110 can directly heat the target region 112. For example, the target region 112 can include the groin of the subject 106. The electromagnetic energy source 108 can output the electromagnetic energy 110 toward the groin. The electromagnetic energy 110 output from the electromagnetic energy source 108 can be absorbed by the groin. As the electromagnetic energy 110 is absorbed, the electromagnetic energy 110 can cause molecules to vibrate, producing heat in the groin that warms the groin. Thus, the electromagnetic energy source 108 can selectively output electromagnetic energy 110 toward the groin to heat the groin of the subject. In an embodiment, the target region 112 can include the palms of the hands of the subject 106. The electromagnetic energy source 108 can output the electromagnetic energy 110 toward the palms of the hands. The electromagnetic energy 110 output from the electromagnetic energy source 108 can be absorbed by the palms of the hands. As the electromagnetic energy 110 is absorbed, the electromagnetic energy 110 can produce heat in the palms of the hands that warms the hands. In an embodiment, the target region 112 can include the abdomen of the subject 106. The electromagnetic energy source 108 can output the electromagnetic energy 110 toward the abdomen. The electromagnetic energy 110 output from the electromagnetic energy source 108 can be absorbed by the abdomen. As the electromagnetic energy 110 is absorbed, the electromagnetic energy 110 can produce heat in the abdomen that warms the abdomen. In an embodiment, the electromagnetic energy 110 can be configured to directly heat a subsurface target region 112. For example, in an embodiment, the electromagnetic energy 110 is configured to penetrate a depth with the subject's body depending on the nature of the tissue being targeted (e.g., fat, muscles, bones, or organs).

In an embodiment, the heat produced by the electromagnetic energy 111 can indirectly heat the target region 112. For example, the target region 112 can include the lungs, brain, or heart of the subject 106. The electromagnetic energy source 108 can selectively output or emit the electromagnetic energy 110 towards the skin surface of the subject 106. The output electromagnetic energy 110 can then be absorbed by the skin, tissue underlying the skin, and one or more major arteries in or near the underlying tissue (e.g., the axillary artery). As the electromagnetic energy 110 is absorbed, the electromagnetic energy 110 produces heat in the skin, underlying tissue, and the one or more major arteries. From the one or more major arteries, blood flow and conductive heat flow can transfer the heat produced by the electromagnetic energy 110 to heat or warm the subject's brain, lungs, or heart, thereby raising the body temperature of the subject 106. In an embodiment, the electromagnetic energy 110 is selected to raise or maintain the body temperature of the subject 106 to above 35° C., above 36° C., or above 37° C. In an embodiment, the electromagnetic energy 110 is selected configured to raise the body temperature of the subject 106 to a temperature between about 35° C. and about 40° C., between about 36° C. and about 39° C., or between about 37° C. and about 38° C. In an embodiment, the electromagnetic energy 110 can directly and indirectly heat the target region 112.

The electromagnetic energy source 108 can include, but is not limited to, a microwave energy source, a radio-frequency energy source, or a magnetic energy source. In an embodiment, the electromagnetic energy source 108 includes a microwave energy source that outputs microwave energy 110 to selectively heat the target region 112. In an embodiment, the microwave energy source 108 includes a steerable microwave energy source. For example, the steerable microwave energy source 108 can include a physically steered or translated microwave energy source 108. In an embodiment, the steerable microwave energy source can include a phased-array microwave energy source 108. In an embodiment, the steerable microwave energy source can include a metamaterial array microwave energy source. For example, the metamaterial array microwave energy source can include a metamaterial surface antenna array commercially available from Kymeta Corporation. In an embodiment, the electromagnetic energy source 108 includes an alternating magnetic field.

In an embodiment, the electromagnetic energy source 108 can include, but is not limited to, a radio-frequency energy source that outputs radio-frequency energy 110 to selectively heat the target region 112. In an embodiment, the electromagnetic energy source 108 can include a magnetic energy source that outputs alternating magnetic energy 110 to selectively heat the target region 112. For example, the electromagnetic energy 110 has a frequency greater than about 1 GHz, about 5 GHz, about 10 GHz, or about 50 GHz. In an embodiment, the electromagnetic energy source 108 includes a plurality of electromagnetic energy sources, such as two or more, or three or more electromagnetic energy sources. In an embodiment, the electromagnetic energy 110 includes multiple types of electromagnetic energy. For example, the electromagnetic energy can include microwave energy, magnetic energy, and light energy. In an embodiment, the electromagnetic energy source 108 can include a plurality of electromagnetic energy sources. For example, the electromagnetic energy source 108 can include a microwave energy source and a radio-frequency energy source. In an embodiment, the electromagnetic energy source 108 can include a microwave energy source and a magnetic energy source. In an embodiment, the electromagnetic energy source 108 can include a radio-frequency energy source and a magnetic energy source. In an embodiment, the electromagnetic energy source 108 can include a microwave energy source, a radio-frequency energy source, and a magnetic energy source. In an embodiment, the electromagnetic energy source 108 can include a plurality of at least one of a microwave energy source, a radio-frequency energy source, or a magnetic energy source. For example, the electromagnetic energy source 108 can include two, three, or any other suitable number of microwave energy sources.

The hypothermia treatment system 100 further includes one or more sensors 114. In the illustrated embodiment, the one or more sensors 114 is coupled (e.g., mounted) to the support structure 102. However, in an embodiment, the one or more sensors 114 are deployed internally within the subject 106. In an embodiment, the one or more sensors 114 are physically coupled to a skin surface of the subject 106. In an embodiment, the one or more sensors 114 are incorporated in the support structure 102.

In an embodiment, the one or more sensors 114 are configured to determine or measure a temperature of the target region 112 of the subject 106. In an embodiment, the one or more sensors 114 determine a temperature of the target region 112 by directly measuring the temperature of the target region 112. In an embodiment, the one or more sensors 114 determine a temperature of the target region 112 by measuring one or more non-target temperatures in a non-target region of the subject 106 and inferring the temperature of the target region 112 from the one or more non-target temperatures. For example, the one or more sensors 114 can be configured to determine the temperature of the esophagus of the subject 106 by measuring the temperature of skin covering the esophagus and inferring the temperature of the esophagus from the temperature of the skin covering the esophagus. In an embodiment, the one or more sensors 114 are configured to determine a temperature of the target region 112 by scanning one or more regions of the subject 106. For example, the one or more sensors 114 can include one or more infrared sensors that sweep across and scan the skin surface on or covering target region 112. In an embodiment, the one or more sensors 114 can include one or more radiometers that sense subsurface target region temperatures.

In an embodiment, the one or more sensors 114 are configured to determine a temperature of the target region 112 before the electromagnetic energy 110 arrives at the target region 112. In an embodiment, the one or more sensors 114 are configured to determine the temperature of the target region 112 after the electromagnetic energy 110 arrives at the target region 112. Accordingly, the electromagnetic energy source 108 and the one or more sensors 114 (e.g., temperature sensors) can be asynchronous so that they do not interfere with one another. In an embodiment, asynchronous operation of the electromagnetic energy source 108 and the one or more sensors 114 can be provided by interleaving operational periods of the electromagnetic energy source 108 with measurement periods of the one or more sensors 114. In an embodiment, the one or more sensors 114 are configured to determine the temperature of the target region 112 simultaneously with the electromagnetic energy 110 arriving at the target region 112. The one or more sensors 114 can include, but are not limited to, fiber-optic temperature sensors, optical coherency tomography sensors, electromagnetic energy detectors, thermography sensors, temperature probes, thermistors, surface temperature sensors, thermobeads, thermopiles, tympanic thermometers, chip-infrared temperature sensors, mini-chip thermistors, thermocouples, clinical thermometers, recording thermometers, rectal thermometers, or resistance thermometers. For example, the one or more sensors 114 can include a thermistor inserted within the target region 112 to measure the temperature of the target region 112. In an embodiment, the one or more sensors 114 can include a radiometer (e.g., using infrared or microwaves) that remotely determines the temperature of the target region 112. The one or more sensors 114 can include a single sensor or a plurality of sensors. The one or more sensors 114 can be small in size, such as a sensor or a sensor array that is a chip-infrared sensor.

In an embodiment, the one or more sensors 114 is configured to determine or measure physiological or other characteristics of the target region 112, which include, but are not limited to, electrical resistivity thereof, blood flow, position, chemical composition thereof, or density thereof. One or more of these sensing capabilities can be present in a single sensor or the array of sensors; sensing capabilities are not limited to a particular number or type of sensors. The one or more sensors 114 can include, but are not limited to, ultrasound sensors, pressure sensors, light sensors, sensors including piezoelectric crystals, encoders, transducers, motion sensors, position sensors, flows sensors, viscosity sensors, shear sensors, time detectors (e.g., timer, clocks), imaging detectors, acoustic sensors, temperature sensors, chemical and biological detectors, electromagnetic energy detectors, pH detectors, or electrical sensors. In an embodiment, the one or more sensors 114 including one or more electromagnetic energy detectors can be configured to determine the absorption, reflection, or emission of the electromagnetic energy 110 in the target region 112. The one or more sensors 114 including a machine-vision system can detect location, quality of location, or quality of output placement of the electromagnetic energy 110 in the target region 112. In an embodiment, the one or more sensors 114, including a contactless, infrared sensor or optical coherency tomography sensor can detect one or more physiological conditions of a subject 106, including, but not limited to, tissue swelling or inflammation.

The hypothermia treatment system 100 further includes a control system 116 including control electrical circuitry (not shown), along with a user interface 118 (e.g., a touchscreen, keypad, etc.) for user input. The control system 116 is operably coupled to the electromagnetic energy source 108 and the one or more sensors 114 to control operation of one or more of the foregoing system components. In an embodiment, the control system 116 is configured to direct the electromagnetic energy source 108 to emit the electromagnetic energy 110 based on feedback or the one or more sensing signals from the one or more sensors 114. For example, in operation, the electromagnetic energy source 108 and the one or more sensors 114 are positioned near or adjacent to the target region 112. One or more sensing signals 120 are output from the one or more sensors 114 to the control system 116 that encodes sensing information. In an embodiment, automatically responsive to the one or more sensing signals 120, the control system 116 outputs one or more emitting information signals 122 to the electromagnetic energy source 108 that encode emitting information or directions. Responsive to the emitting information signals 122, the electromagnetic energy source 108 can emit the electromagnetic energy 110 toward the target region 112 of the subject 106. The control system 116 can also output one or more sensing instructions to the one or more sensors 114. In an embodiment, the one or more sensors 114 determine the temperature of the target region 112 in accordance with the sensing instructions. Thus, in an embodiment, the control system 116 is configured to control operation of both the electromagnetic energy source 108 and the one or more sensors 114. In an embodiment, the control system 116 is wirelessly connected to the electromagnetic energy source 108 and the one or more sensors 114. In an embodiment, the control system 116 can adjust the output of the electromagnetic energy 110 from the electromagnetic energy source 108 to achieve a substantially uniform temperature of the target region 112. For example, in an embodiment, the electromagnetic energy source 108 can include an antenna system and the control system 116 can control the antenna system of the electromagnetic energy source 108 to continuously shift the electromagnetic energy 110 to achieve a substantially uniform temperature of the target region 112. In an embodiment, the control system 116 can control the antenna system to continuously shift the electromagnetic energy 110 in one or more patterns. In an embodiment, the electromagnetic energy source 108 can include a scanning system and the control system 116 can control the scanning system of the electromagnetic energy source 108 to continuously shift the electromagnetic energy 110 to achieve a substantially uniform temperature of the target region 112. In an embodiment, the electromagnetic energy source 108 is configured to pulse the target region 112 with the electromagnetic energy 110.

In an embodiment, the control system 116 is configured to control at least one operational parameter of the electromagnetic energy source 108 to achieve a uniform temperature of the target region 112. For example, the one or more emitting information signals 122 can include one or more directions to emit the electromagnetic energy 110 toward the target region 112 as the electromagnetic energy source 108 moves over the target region 112. As another example, the one or more emitting information signals 122 can include one or more locations of the electromagnetic energy 110. In an embodiment, the one or more emitting information signals 122 can include one or more directions to emit the electromagnetic energy 110 toward a portion of the target region 112 determined to have a temperature lower than another portion of the target region 112.

In an embodiment, the one or more emitting information signals 122 include one or more directions to emit the electromagnetic energy 110 after the one or more sensors 114 determine the temperature of the target region 112. In an embodiment, the one or more emitting information signals 122 include one or more directions to stop emitting the electromagnetic energy 110 after the one or more sensors 114 determine the temperature of the target region 112. In an embodiment, the one or more emitting information signals 122 include one or more directions to emit the electromagnetic energy 110 substantially and simultaneously with the electromagnetic energy source 108 emitting the electromagnetic energy 110 toward the target region 112.

In an embodiment, the one or more emitting information signals 122 include one or more directions to control at least one operational parameter of the electromagnetic energy source 108. The one or more emitting information signals 122 can include one or more directions to control the intensity of the electromagnetic energy 110. In an embodiment, the one or more emitting information signals 122 can include one or more directions to control the duration of the electromagnetic energy 110 applied to the target region 112. In an embodiment, the one or more emitting information signals 122 can include one or more directions to control the direction of the electromagnetic energy 110 emitted from the electromagnetic energy source 108. In an embodiment, the one or more emitting information signals 122 can include one or more directions to control the type of electromagnetic energy 110. In an embodiment, the one or more emitting information signals 122 can include one or more directions to control the phase of electromagnetic energy 110. In an embodiment, the one or more emitting information signals 122 can include one or more directions to control the frequency of electromagnetic energy 110. In an embodiment, the one or more emitting information signals 122 can include one or more directions to control the pulse frequency of the electromagnetic energy 110 emitted from the electromagnetic energy source 108. In an embodiment, the one or more emitting information signals 122 can include one or more directions to control the position of the electromagnetic energy source 108. In an embodiment, the one or more emitting information signals 122 can include one or more directions to control the alignment of the electromagnetic energy source 108 with the target region 108. In an embodiment, the one or more emitting information signals 122 include one or more directions to control the electromagnetic energy source 108 in order to vary the electromagnetic absorption profile within the subject 106. In an embodiment, the one or more emitting information signals 122 include one or more directions to emit the electromagnetic energy 110 in one or more timed intervals. For example, the time intervals include, but are not limited to, fixed timed intervals, periodic time intervals (e.g. pulses), programmable or programmed time intervals, triggered time intervals, manually determined time intervals, automatic time intervals, remotely controlled time intervals, or time intervals based on feedback from the one or more sensors 114. In an embodiment, the one or more emitting information signals 122 include one or more directions to aim the electromagnetic energy 110 toward the target region 112. In an embodiment, the one or more emitting information signals 122 include one or more directions to move the electromagnetic energy source 108 toward the target region 112. In an embodiment, the one or more emitting information signals 122 include one or more directions to align the electromagnetic energy 110 with the target region 112.

In an embodiment, the one or more emitting information signals 122 include one or more directions to emit the electromagnetic energy 110 in one or more pulses. For example, the one or more emitting information signals 122 include one or more directions to direct the electromagnetic energy source 108 to emit a first electromagnetic energy and, before the electromagnetic energy source 108 stops emitting the first electromagnetic energy, the one or more directions direct the electromagnetic energy source 108 to emit a second electromagnetic energy. The one or more emitting information signals 122 can also include one or more directions to stop emitting the first electromagnetic energy and to emit a third electromagnetic energy. In an embodiment, the first electromagnetic energy, the second electromagnetic energy, or the third electromagnetic energy are different.

In an embodiment, the one or more emitting information signals 122 include one or more directions to stop emitting the electromagnetic energy 110 if one or more conditions are detected. For example, the one or more emitting information signals 122 can include one or more directions to stop emitting the electromagnetic energy 110 if the temperature of the target region 112 reaches an upper threshold temperature. In an embodiment, the upper threshold temperature is any temperature below about 40° C., about 42° C., about 44° C., about 46° C. or about 48° C. In an embodiment, the upper threshold temperature is any temperature between about 40° C. and about 49° C. or between about 40.5° C. and about 45° C. The upper threshold temperature may vary depending on the target region 112 or tissue type (fat, muscles, bones, normal tissue, etc.). The upper threshold temperature may vary depending on the thermal history of the target region 112 or on a length of time at which the temperature of the target region 112 will be at or exceed the upper threshold temperature.

In an embodiment, the upper threshold temperature is selected to be below a tissue damaging temperature. For example, the tissue damaging temperature can be any temperature above about 40° C., about 42° C., about 44° C., about 46° C. or about 48° C. In an embodiment, the tissue damaging temperature is any temperature between about 40° C. and about 49° C. or between about 40.5° C. and about 45° C. The tissue damaging temperature may vary depending on the target region or type of tissue. In an embodiment, the tissue damaging temperature is associated with apoptosis. In an embodiment, the tissue damaging temperature is associated with necrosis. In an embodiment, the tissue damaging temperature is associated with mitotic catastrophe. In an embodiment, the tissue damaging temperature is associated with senescence. In an embodiment, the tissue damaging temperature is associated with autophagy. The tissue damaging temperature may vary depending on the target region, thermal history, or type of tissue.

In an embodiment, the one or more emitting information signals 122 can include one or more directions to emit the electromagnetic energy 110 if the temperature of the target region 112 or the body temperature of the subject 106 falls below a lower threshold temperature. In an embodiment, the lower threshold temperature is any temperature below about 35° C. In an embodiment, the lower threshold temperature is any temperature below about 34° C. In an embodiment, the lower threshold temperature is any temperature below about 33° C. In an embodiment, the lower threshold temperature is between about 20° C. and about 35° C., about 22° C. and about 33° C., about 24° C. and about 31° C., or about 25° C. and about 30° C.

In an embodiment, the control system 116 includes processing hardware (e.g., processing electrical circuitry) and an operating system configured to run one or more application software programs. In an embodiment, the control system 116 can use one or more processing techniques on the one or more sensing signals 120 to determine at least location, direction, movement, or presence of the electromagnetic energy 110. For example, an analysis of the one or more sensing signals 120 can generate distances to the one or more sensors 114. From determined temperatures and the distances to the one or more sensors 114, spatial information (e.g., position, three-dimensional position, distribution, presence) of the electromagnetic energy 110 can be determined by the control system 116. In an embodiment, the control system 116 can send one or more instructions to the electromagnetic energy source 108 to emit and direct the electromagnetic energy 110 at a colder portion of the target region 112 to heat the colder portion or bring the temperature of the colder portion into uniformity with other portions of the target region 112. In addition to determining spatial information, the control system 116 can determine motion information for the electromagnetic energy 110 based on the one or more sensing signals 120 received from the one or more sensors 114.

In an embodiment, the processing hardware or processor numerically models electromagnetic propagation. For example, the control system 116 can use one or more processing techniques on the one or more sensing signals 120 to numerically model electromagnetic propagation. In an embodiment, the processing hardware or processor numerically models energy absorption. For example, the control system 116 can use one or more processing techniques on the one or more sensing signals 120 to numerically model energy absorption. In an embodiment, the control system 116 determines an electromagnetic absorption pattern for the target region 112 at least partially based on the one or more sensing signals 120 including physiological data of the subject 106. In an embodiment, the electromagnetic energy pattern is determined by forming a three-dimensional pattern directly from the one or more sensing signals 120 including physiological data of the subject 106. In an embodiment, the control system 116 directs the one or more sensors 114 to scan a focal region of the target region 112. The electromagnetic energy pattern is determined from the one or more sensing signals 120. In an embodiment, the processing hardware or processor numerically models thermal transport within the subject 106. The numerical model of thermal transport can include the effects of thermal transport via blood flow and thermal transport via thermal conduction and diffusion within tissue. For example, the control system 116 can use one or more processing techniques on the one or more sensing signals 120 to numerically model thermal transport. In an embodiment, the control system 116 can use one or more processing techniques to numerically model thermal transport within the subject 106 resulting from absorption of the electromagnetic energy 110. In an embodiment, the control system 116 controls the electromagnetic energy source 108 based on real time feedback from an output from any of the numerical models generated by the processing hardware or processor.

In an embodiment, the control system 116 uses computational analysis to generate an electromagnetic energy irradiation profile to treat the subject 106 for hypothermia. In an embodiment, the control system 116 uses computational analysis to generate the electromagnetic energy irradiation profile at least partially based on feedback from the one or more sensors 114. For example, the control system 116 uses computational analysis to generate the electromagnetic energy irradiation profile at least partially based on the one or more sensing signals 120 from one or more sensors 114 including one or more electromagnetic energy detectors. In an embodiment, the control system 116 uses computational analysis to select an amount of electromagnetic energy 110 to treat the subject 106 based on feedback from the one or more sensors 114. For example, the control system 116 can use computational analysis to select an amount of electromagnetic energy 110 to treat the subject 106 based on the one or more sensing signals 120 from the one or more sensors 114 including one or more skin temperature sensors.

Referring to FIG. 1B, in an embodiment, the hypothermia treatment system 100 includes medical imaging equipment 124 operably associated with the control system 116. In the illustrated embodiment, the medical imaging equipment 124 is incorporated in the support structure 102. However, in an embodiment, the medical imaging equipment 124 is positioned under the support structure 102. In an embodiment, the medical imaging equipment 124 is positioned over the support structure 102. In an embodiment, the medical imaging equipment 124 is configured to determine subject-specific body data. In an embodiment, the subject-specific body data includes one or more physiological parameters of the subject 106. The one or more physiological parameters can include, but is not limited to, temperature, body temperature, peripheral temperatures, heart rate, blood pressure, blood flow, respiration, blood volume, shivering, physiological electrical fields, electromagnetic energy radiation levels, tissue density, body shape, or movement. The medical imaging equipment 124 can include, but is not limited to, a magnetic resonance imaging device or a computed tomography system. For example, the medical imaging equipment 124 can include a magnetic resonance imaging device that produces anatomical images of the subject 106 in a plurality of different orientations.

In operation, one or more physiological information signals 126 are output from the medical imaging equipment 124 to the control system 116 that encodes body-specific or physiological data. In an embodiment, responsive to the physiological information signals 126, the control system 116 outputs one or more emitting information signals 122 to the electromagnetic energy source 108. Responsive to the emitting-information signals 122, the electromagnetic energy source 108 can emit the electromagnetic energy 110 toward the target region 112 of the subject 106. The control system 116 can also output one or more instructions to the medical imaging equipment 124. In an embodiment, the medical imaging equipment 124 can determine physiological parameters in accordance with the instructions from the control system 116. In an embodiment, the control system 116 can use one or more processing techniques on the one or more physiological information signals 126 to generate an electromagnetic irradiation information profile within the target region 112. For example, an analysis of the one or more physiological information signals 126 can generate the level of electromagnetic energy at different locations within the target region 112. From the electromagnetic energy levels and locations, an electromagnetic irradiation information profile of the target region 112 can be determined by the control system 116. Based on an electromagnetic irradiation information profile of the target region 112, the control system 116 can control at least one operational parameter of the electromagnetic energy source 108, such as output or movement. In an embodiment, the control system 116 controls the electromagnetic energy source 108 based on real time feedback from the medical imaging equipment 124. In an embodiment, the control system 116 controls the electromagnetic energy source 108 based on previously received feedback from the medical imaging equipment 124.

In an embodiment, the control system 116 determines an electromagnetic absorption pattern for the target region 112 at least partially based on the one or more physiological information signals 126 from the medical imaging equipment 124. In an embodiment, the electromagnetic energy pattern is determined by forming a three-dimensional pattern directly from the one or more physiological information signals 126. In an embodiment, the control system 116 directs the medical imaging equipment 124 to scan a focal region of the target region 112 and the electromagnetic energy pattern is determined from the one or more physiological information signals 126.

Referring to FIG. 1C, in an embodiment, the one or more sensors 114 are included in a thermography system 128 operably coupled to the control system 116. In the illustrated embodiment, the thermography system 128 is incorporated in the support structure 102. However, in an embodiment, the thermography system 128 is positioned under the support structure 102. In an embodiment, the thermography system 128 is remote from the electromagnetic energy source 108.

In an embodiment, the thermography system 128 is configured to determine a temperature distribution in a subsurface target region 112. In an embodiment, the thermography system 128 is configured to determine a temperature distribution in a surface target region 112. In an embodiment, the thermography system 128 can provide feedback control over the electromagnetic energy source 108. For example, the control system 116 can direct the thermography system 128 to determine a temperature profile in the target region 112.

In an embodiment, the thermography system 128 includes an antenna 156 and a transceiver (not shown) or a transmitter and a receiver (not shown). In an embodiment, the transceiver can supply electric current to the antenna 156 and the antenna 156 can radiate energy from the current as electromagnetic waves. In reception, the antenna 156 can receive electromagnetic energy and produce a voltage that is converted into one or more thermography signals 130. Optionally, the voltage is applied to the transceiver to be amplified. In an embodiment, the one or more sensors 114 can share one or more components with the antenna 156. For example, in an embodiment, the one or more sensors 114 and the antenna 156 can share a transmitter. In an embodiment, the antenna can be incorporated in the one or more sensors 114.

The one or more thermography signals 130 can be output from the thermography system 128 to the control system 116. In an embodiment, the one or more thermography signals 130 can include temperature profile information. Responsive to the feedback from the thermography system 128, the control system 116 can control at least one operational parameter of the electromagnetic energy source 108. For example, if the temperature profile information indicates a portion of the target region 112 exhibits a lower temperature than other portions of the target region 112, the control system 116 can direct the electromagnetic energy source 108 to align the electromagnetic energy 110 with the lower temperature portion and emit the electromagnetic energy 110 toward the lower temperature portion. In an embodiment, based on the temperature profile information, the control system 116 can direct the electromagnetic energy source 108 to scan the target region 112.

In an embodiment, the control system 116 can control the electromagnetic energy source 108 based on real time feedback from the thermography system 128. In an embodiment, the control system 116 can calibrate the electromagnetic energy source 108 based on previously received feedback from the thermography system 128. For example, the control system 116 can receive one or more thermography signals 130 from the thermography system 128 that includes temperature profile information. Based on the temperature profile information, the control system 116 can calibrate an electromagnetic energy deposition profile of the electromagnetic energy source 108. In an embodiment, the control system 116 can calibrate the amount of electromagnetic energy 110 emitted or outputted from the electromagnetic energy source 108.

In an embodiment, the thermography system 128 includes a thermographic camera. In an embodiment, the thermography system 128 includes microwave energy. In an embodiment, the thermography system 128 includes a microwave radiometer. In an embodiment, the thermography system 128 includes a magnetic resonance imaging system. In an embodiment, the thermography system 128 includes a radiography system. In an embodiment, the thermography system 128 includes invasive probes. In an embodiment, the thermography system 128 uses radiography. In an embodiment, the thermography system 128 includes particles carried in the blood of the subject 106 with temperature-dependent electromagnetic (e.g., microwave energy) properties. In an embodiment, the thermography system includes temperature-dependent ultrasound contrast agents.

In an embodiment, the control system 116 is configured to determine an electromagnetic absorption pattern for the target region 112 at least partially based on the one or more thermography signals 130 from the thermography system 128. In an embodiment, the control system 116 determines the electromagnetic absorption pattern by forming a three-dimensional pattern directly from the one or more thermography signals 130. In an embodiment, the control system 116 directs the thermography system 128 to scan a focal region of the target region 112 and the electromagnetic absorption pattern is determined from the one or more thermography signals 130. In an embodiment, the control system 116 or the thermography system 128 scans the electromagnetic absorption pattern using thermal inertia of the tissue of the target region 112 as a thermal ballast.

In an embodiment, the control system 116 uses computational analysis to select or simulate an electromagnetic energy irradiation profile to treat the subject 106 for hypothermia. In an embodiment, the control system 116 uses computational analysis to select or simulate an electromagnetic energy irradiation profile from a database of electromagnetic energy irradiation profiles. In an embodiment, the control system 116 uses computational analysis to select or simulate an electromagnetic energy irradiation profile at least partially based on feedback from the thermography system 128. For example, the control system 116 can select the electromagnetic energy irradiation profile at least partially based on subject-specific body data from a magnetic resonance imaging system.

In an embodiment, the control system 116 uses computational analysis to select an amount or type of electromagnetic energy 110 to treat the subject 106 for hypothermia. In an embodiment, the control system 116 uses computational analysis to select an amount of electromagnetic energy 110 to treat the subject 106 from one or more electromagnetic energy dosage tables. In an embodiment, the control system 116 uses computational analysis to select an amount of electromagnetic energy 110 to treat the subject 106 from a database. In an embodiment, the control system 116 uses computational analysis to select a type of electromagnetic energy (e.g., microwave, radio frequency, or alternating magnetic field) to treat the subject 106 for hypothermia from a database of properties of electromagnetic energy. In an embodiment, the control system 116 uses computational analysis to select an amount or type of electromagnetic energy 110 based on feedback from the thermography system 128. For example, the control system 116 can use computational analysis to select an amount or type of electromagnetic energy 110 based subject-specific body data from a computed tomography scan.

Referring to FIG. 1D, in an embodiment, the hypothermia treatment system 100 includes a reflective material 132. In the illustrated embodiment, the reflective material 132 is a reflective patient covering positioned over the subject 106. However, in an embodiment, the reflective material 132 is positioned under the subject 106, such as the reflective material 132 can be disposed on a portion of the support structure 102 on which the subject 106 rests or form part of the support structure 102 on which the subject 106 rests. For example, the electromagnetic energy source 108 can be positioned over the subject 106 and the reflective material 132 can be incorporated in the support structure 102. In an embodiment, the reflective material 132 extends around one or more portions of the subject 106. For example, the reflective material 132 can at least partially extend around one or more legs of the subject 106. In an embodiment, the reflective material 132 can at least partially extend around the groin of the subject 106. In an embodiment, the reflective material 132 can at least partially extend around the back or abdomen of the subject 106.

The reflective material 132 can exhibit a variety of different configurations selected for a particular application. For example, the reflective material 132 can be configured as a blanket, a sheet, a surgical gown, or a pad. In an embodiment, the reflective material 132 can be sized to generally correspond to the body of the subject 106. However, in an embodiment, the reflective material 132 can be sized to generally correspond to the size of one or more portions of the target region 112. In an embodiment, the reflective material 132 can be configured to leave a surgical region on the subject 106 uncovered by the reflective material 132. In an embodiment, the reflective material 132 can be sized proportional to the size of the target region 112. For example, the reflective material 132 can exhibit a lateral dimension that is between about 1.1 and about 3 times greater than a lateral dimension of the target region 112. In an embodiment, the reflective material 132 can exhibit a lateral dimension that is between about 1.2 and about 2.5 times greater than a lateral dimension of the target region 112. The reflective material 132 can include, but is not limited to, mylar, aluminum, reflective fabric, metallic foil, silver, or gold. In an embodiment, the reflective material 132 can include two or more layers of material. In an embodiment, the reflective material 132 can include different reflective materials. In an embodiment, the reflective material 132 can be configured as an aerosol, including reflective particles that is sprayable onto a skin surface of the subject 106 over the target region 112. In an embodiment, the reflective material 132 can be configured as a coating.

In an embodiment, the reflective material 132 is configured to at least partially control irradiation of the electromagnetic energy 110. For example, the reflective material 132 can be positioned over the subject 106 supported on the support structure 102. The reflective material 132 and the support structure 102 can form a containment area for containing the electromagnetic energy 110. As the electromagnetic energy source 108 outputs the electromagnetic energy 110 into the containment area, the reflective material 132 can reflect the electromagnetic energy 110 within the containment area back toward the subject 106 or electromagnetic energy 108 to limit leakage of the electromagnetic energy 110 therefrom. In an embodiment, the containment area can be configured to contain, intercept, or trap more than about 80%, about 85%, about 90%, about 95%, or 99% of the electromagnetic energy 110 irradiating away from the subject 106. In an embodiment, the reflective material 132 can at least partially control irradiation of the electromagnetic energy 110 by reflecting electromagnetic energy 110 that passes through the subject 106 back into or through the subject 106. For example, the reflective material 132 can reflective the electromagnetic energy 110 toward the target area 112. As the reflected electromagnetic energy 110 passes into the subject 106 over the target region 112, the reflected electromagnetic energy 110 can be absorbed by the subject 106, thereby producing heat to further heat the target region 112. In an embodiment, the reflective material 132 can also be configured to impede heat transfer via conduction or convection. For example, the reflective material 132 can include two or more layers spaced apart by one or more gaps therebetween.

In an embodiment, the reflective material 132 can be configured to shield medical personnel from the electromagnetic energy 110. For example, in an embodiment, the reflective material 132 can be configured to direct or reflect electromagnetic energy 110 away from medical personnel including nurses, doctors, or technicians. In an embodiment, the reflective material 132 can be configured as a bag and the subject 106 and the electromagnetic energy source 108 are positionable within the bag.

Referring to FIG. 1E, in an embodiment, the hypothermia treatment system 100 includes an energy absorptive material 134. In the illustrated embodiment, the energy absorptive material 134 is positioned between the subject 106 and the support structure 102. However, in an embodiment, the energy absorptive material 134 is incorporated in the support structure 102. In an embodiment, the energy absorptive material 134 can include a patient covering. The energy absorptive material is sized similar to the reflective material 132. For example, the energy absorptive material 134 can be sized to generally correspond to the size of the target region 112. In an embodiment, the energy absorptive material 134 is configured to leave a surgical region on the subject 106 uncovered by the energy absorptive material 134. The energy absorptive material 134 can include, but is not limited to, composite materials, ceramic materials, multilayer insulation materials, nonlinear magnetic materials, iron, graphite, or lead. In an embodiment, the energy absorptive material 134 can include two or more layers of material.

The energy absorptive material 134 can be configured to at least partially control irradiation of the electromagnetic energy 110. For example, the energy absorptive material 134 can be distributed to distribute heat over the target region 112. In an embodiment, the energy absorptive material 134 can enhance heat generated from the electromagnetic energy 110. In an embodiment, the energy absorptive material 134 is configured to shield medical personnel from the electromagnetic energy 110. For example, in an embodiment, the energy absorptive material 134 is positioned between the electromagnetic energy source 108 and medical personnel including doctors, nurses, or technicians.

In an embodiment, energy absorptive material 134 includes different energy absorptive materials. In an embodiment, the energy absorptive material 134 is configured as an aerosol spray including absorptive particles that can be deposited onto a skin surface of the subject 106 over the target region 112. In an embodiment, the energy absorptive material 134 is configured as a coating.

In any of the disclosed hypothermia treatment systems, the hypothermia treatment system can include an array of sensors associated with an electromagnetic energy source to determine one or more parameters. For example, referring to the embodiment shown in FIG. 2A, an array of sensors 114a-114n are operably associated with the electromagnetic energy source 108. In the illustrated embodiment, the array of sensors 114a-114n is incorporated in the support structure 102. However, in an embodiment, the array of sensors 114a-114n is physically coupled to a skin surface of the subject 106. In an embodiment, each of the sensors 114a-114n is respectively operably coupled to the electromagnetic energy source 108.

The array of sensors 114a-114n can include any suitable sensors configured to determine a temperature of the target region 112. For example, one or more of the array of sensors 114a-114n can include, but are not limited to, fiber optic temperature sensors, thermography sensors, temperature probes, thermistors, surface temperature sensors, thermobeads, thermopiles, tympanic thermometers, infrared temperature sensors, mini-chip thermistors, and thermocouples, clinical thermometers, recording thermometers, rectal thermometers, and resistance thermometers. In addition, the array of sensors 114a-114n can include other types of sensors, such as, for example, ultrasound sensors, pressure sensors, light sensors, sensors including piezoelectric crystals, encoders, transducers, motion sensors, position sensors, flow sensors, viscosity sensors, shear sensors, time detectors (e.g., timer, clocks), imaging detectors, acoustic sensors, temperature sensors, chemical and biological detectors, electromagnetic energy detectors (e.g., optical energy such as near IR, UV, visual), pH detectors, or electrical sensors. The array of sensors 114a-114n can be configured to determine various other characteristics of the target region 112, such characteristics including, but not limited to, electrical resistivity thereof, position, chemical composition thereof, or density thereof. One or more of these sensing capabilities can be present in a single sensor or the array of sensors 114a-114n; sensing capabilities are not limited to a particular number or type of sensors.

In an embodiment, the array of sensors 114a-114n detects temperature over an area of the body of the subject 106, such as the target region 112, to facilitate the determination of a temperature gradient or profile. The array of sensors 114a-114n covert thermal energy to one or more sensing signals 120 in the form of electrical energy. In an embodiment, one or more analog-to-digital converters (ADC) convert the electrical energy to digital data that is sent to the control system 116. The ADC can be a separate component, can be integrated into the control system 116, or can be integrated into the array of sensors 114a-114n. In an embodiment, the control system 116 includes processing hardware (e.g., processing electrical circuitry) and an operating system configured to run one or more application software programs. The control system 116 can use one or more processing techniques to analyze the digital data in order to determine different parameters, including temperature gradient, position of the target region 112 or electromagnetic energy 110, temperature profile of the target region 112, or electromagnetic radiation profile of the target region 112.

In any of the disclosed hypothermia treatment systems, the hypothermia treatment system can include a plurality of electromagnetic energy sources associated with one or more sensors. For example, referring to the embodiment shown in FIG. 2B, a plurality of electromagnetic energy sources 108a-108n are operably associated with an array of sensors 114a-114n and the control system 116. In the illustrated embodiment, the plurality of electromagnetic energy sources 108a-108n are positioned below the support structure 102. However, in an embodiment, the plurality of electromagnetic energy sources 108a-108n are incorporated in the support structure 102. The plurality of electromagnetic energy sources 108a-108n can include any suitable electromagnetic energy source. For example, one or more of the plurality of electromagnetic energy sources 108a-108n can include, but are not limited to, a microwave energy source, a radio-frequency source, or a magnetic energy source. In an embodiment, the electromagnetic energy sources 108a-108n are the same as one another. For example, each of the electromagnetic energy sources 108a-108n can include a microwave energy source. In an embodiment, the electromagnetic energy sources 108a-108n are different from one another. For example, the electromagnetic energy sources 108a-108n can include a microwave energy source and a radio-frequency energy source.

In an embodiment, the array of sensors 114a-114n detects temperature over an area of the body of the subject 106, such as a target region including target regions 112a-112n. For example, the array of sensors 114a-114n can convert thermal energy to one or more sensing signals 120 which are then sent to the control system 116 by the array of sensors 114a-114n. The control system 116 can use one or more processing techniques to analyze the one or more sensing signals 120 to determine one or more different parameters, including, but not limited to, temperature gradient, position of the target regions 112a-112n or electromagnetic energy 110, temperature profile of the target regions 112a-112n, or electromagnetic radiation profile of the target regions 112a-112n. In an embodiment, the electromagnetic energy sources 108a-108n are controlled together, individually, or in one or more groups by the control system 116. The control system 116 can output one or more emitting information signals 122 to the electromagnetic energy sources 108a-108n based on the parameters determined by the control system 116, such as a temperature gradient or profile. The one or more emitting information signals 112 can include one or more directions to emit electromagnetic energy 110 from one or more of the electromagnetic energy sources 108a-108n. In an embodiment, the one or more emitting information signals 112 can include one or more directions to aim or move one or more of the electromagnetic energy sources 108a-108n toward the target regions 112a-112n.

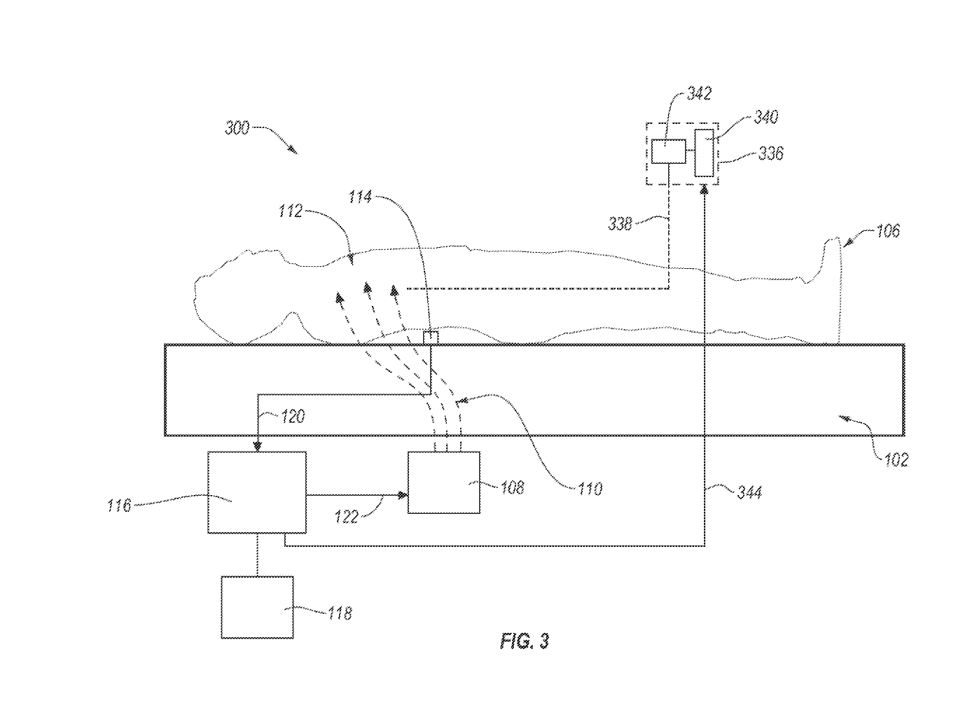

FIG. 3 is a schematic diagram of an embodiment of a hypothermia treatment system 300 including a supply 336 of electromagnetic energy absorption agent 338. The hypothermia treatment system 300 includes many of the same components as the hypothermia treatment system 100 shown in FIGS. 1A through 2B. Therefore, in the interest of brevity, components of the hypothermia treatment system 300 that are identical or similar to each other have been provided with the same reference numerals, and an explanation of their structure and function will not be repeated unless the components function differently in the hypothermia treatment systems 100 and 300. However, it should be noted that the principles of the hypothermia treatment system 300 are employed with any of the embodiments described with respect to FIGS. 1A through 2.

The hypothermia treatment system 300 can include a support structure 102 configured to support a subject 106. The hypothermia treatment system 300 further includes an electromagnetic energy source 108 positioned under the support structure 102. The electromagnetic energy source 108 is configured to selectively output electromagnetic energy 110 toward a target region 112 of the subject 106 to heat the target region 112. Heating the target region 112 with the electromagnetic energy 110 can increase the core body temperature of the subject 106 via thermal conduction, thermal convection, or thermal radiation. In an embodiment, the target region 112 can include one or more locations on or within the body of the subject 106. The electromagnetic energy source 108 can include, but is not limited to, a microwave energy source, a radio-frequency energy source, or a magnetic energy source. The hypothermia treatment system 300 further includes one or more sensors 114 that are configured to determine a temperature or other characteristics of the target region 112 of the subject 106. The hypothermia treatment system 300 further includes a control system 116 including control electrical circuitry (not shown). The control system 116 is operably coupled to the electromagnetic energy source 108, the one or more sensors 114, and a supply 336 of electromagnetic energy absorption agent 338 to control operation of one or more of the foregoing system components.

The supply 336 of electromagnetic energy absorption agent 338 is delivered to the subject 106 to absorb the electromagnetic energy 110. For example, the electromagnetic energy absorption agent 338 can be delivered to the target region 112 and the electromagnetic energy 110 can be delivered to and absorbed by the electromagnetic energy absorption agent 338 to at least partially heat the target region 112. In an embodiment, absorption of the electromagnetic energy 110 by the electromagnetic energy absorption agent 338 can be temperature-dependent. In an embodiment, the electromagnetic energy absorption agent 338 can absorb the electromagnetic energy 110 at a target temperature. For example, the electromagnetic energy absorption agent 338 can include one or more magnetic particles or ferromagnetic particles and the target temperature can include a selected curie temperature. The curie temperature is the temperature of the reversible ferromagnetic or paramagnetic transition of the magnetic particles. Below this temperature, the magnetic particles heat in the electromagnetic energy 110 (e.g., an alternating magnetic field). However, above the Curie Temperature, the magnetic particles become paramagnetic and their magnetic domain becomes unresponsive to the electromagnetic energy 110. In an embodiment, the electromagnetic energy absorption agent 338 can include one or more antiferromagnetic or ferromagnetic particles and the target temperature can include a selected Néel temperature. In an embodiment, the target temperature can include a temperature or thermal profile. In an embodiment, the energy absorption agent 338 can include nanomagnetic material.

In an embodiment, the electromagnetic energy absorption agent 338 can include liquids, solutions, suspensions, mixtures, mist, reagents, micro-particles, molecules, emulsions, or any other fluids suitable to be administered to the subject 106. In an embodiment, the electromagnetic energy absorption agent 338 can include one or more particles. In an embodiment, the one or more particles can include non-bound, blood-carried particles. For example, the electromagnetic energy 110 can be deposited within the non-bound, blood-carried particles within the target region 112 to heat the target region 112. In an embodiment, the particles are incorporated with red blood cells. In an embodiment, the particles are incorporated with ghost cells. In an embodiment, the particles are incorporated with liposomes. In an embodiment, the particles are smaller than 1 μm, and can be absorbed one or more body organs (e.g., liver, spleen, the kidneys, or the lungs). For example, in an embodiment the particles can include ferrite particles.

In an embodiment, the one or more particles can exhibit selective temperature-dependent absorption to deposit the electromagnetic energy 110 into or on the subject 106 or the target region 112. In an embodiment, the one or more particles can exhibit selective temperature dependent electric absorption to deposit the electromagnetic energy 110 into or on the subject 106 or the target region 112. In an embodiment, the one or more particles exhibit selective temperature dependent magnetic absorption to deposit the electromagnetic energy 110 into the subject 106 or the target region 112.

In an embodiment, the electromagnetic energy absorption agent 338 can include metallic particles. In an embodiment, the electromagnetic energy absorption agent 338 can include magnetic particles. In an embodiment, the magnetic particles can include iron oxide. In an embodiment, the magnetic particles can include an iron-nickel alloy. In an embodiment, the magnetic particles can exhibit a curie temperature below an ablation temperature (e.g., 40° C.) of the target region 112. In an embodiment, the electromagnetic energy absorption agent 338 can exhibit a peak absorption temperature below 40° C.

The electromagnetic energy absorption agent 338 can be delivered to the target region 112 orally, topically, via inhalation, via injection, via implantation, or another suitable delivery method. In an embodiment, the electromagnetic energy absorption agent 338 can include nanoparticles, such as, for example, spheres, rods, and shells. In an embodiment, the nanoparticles can include gold nanoparticles.

In an embodiment, the supply 336 of the electromagnetic energy absorption agent 338 can include one or more containers 340 that hold one or more different electromagnetic energy absorption agents 338. The one or more containers 340 can be operably coupled to a delivery unit 342. In an embodiment, the delivery unit 342 can include at least one of a fluid dispensing unit, a force generating mechanism, an actuator, a piston, a pump (e.g., a mechanical pump, or an electro-mechanical pump), or another suitable delivery device. For example, the delivery unit 342 can include at least one of a pneumatic actuator, a hydraulic actuator, a piezoelectric actuator, a linear actuator, an electromechanical actuator, or another suitable actuator for actuating a pump or other device for delivering the electromagnetic energy absorption agent 338. The delivery unit 342 is configured to deliver the electromagnetic energy absorption agent 338 to the subject 106. In an embodiment, the delivery unit 342 is configured to deliver the electromagnetic energy absorption agent 338 into the subject 106. In an embodiment, the delivery unit 342 is configured to deliver the electromagnetic energy absorption agent 338 into a bloodstream of the subject 106. In an embodiment, the delivery unit 342 is configured to deliver the electromagnetic energy absorption agent 338 to the subject intravenously, intramuscularly, or intra-arterially, or subcutaneously. In an embodiment, the delivery unit 342 is configured to deliver the electromagnetic energy absorption agent 338 to the subject orally. In an embodiment, the delivery unit 342 is configured to deliver the electromagnetic energy absorption agent 338 via inhalation or topically. In an embodiment, the delivery unit 342 is configured to deliver the electromagnetic energy absorption agent 338 to the subject 106 rectally. In an embodiment, the delivery unit 342 is configured to deliver the electromagnetic energy absorption agent 338 to the subject via the urethra of the subject 106.

In an embodiment, the one or more containers 340 are individually, operably coupled to the delivery unit 342 via conduits or tubing and corresponding electronically controlled valves (not shown) that can be selectively opened and closed via one or more control signals from the control system 116 to allow the electromagnetic energy absorption agent 338 to be selectively delivered by the delivery unit 342 from the one or more containers 340.

In an embodiment, the control system 116 can output one or more delivery information signals 344 to the supply 336 that encodes delivery information or directions. Responsive to the one or more delivery information signals 344, the delivery unit 342 of the supply 336 can deliver the electromagnetic energy absorption agent 338 from the one or more containers 340 to the subject 106. The control system 116 can also output emitting information signals 122 to the electromagnetic energy source 108. Thus, in an embodiment, the control system 116 is configured to control operation of the supply 336 and the electromagnetic energy source 108. In an embodiment, the delivery information includes information that the electromagnetic energy source 108 is going to output or emit the electromagnetic energy 110. In an embodiment, the one or more delivery information signals 344 include one or more directions to deliver the electromagnetic energy absorption agent 338 internally within the subject 106. In an embodiment, the one or more delivery information signals 344 include one or more directions to deliver the electromagnetic energy absorption agent 338 simultaneously with the electromagnetic energy source 108 emitting the electromagnetic energy 110.

In an embodiment, the one or more emitting information signals 122 can include one or more directions to emit the electromagnetic energy 110 after the delivery unit 342 delivers the electromagnetic energy absorption agent 338. In an embodiment, the one or more emitting information signals 122 can include one or more directions to aim or move the electromagnetic energy source 108 toward the electromagnetic energy absorption agent 338 within or on the subject 106.

In operation, the subject 106 is positioned on the support structure 102. The electromagnetic energy absorption agent 338 is delivered to the target region 112 of the subject 106 from the supply 336 (under the control of the control system 116). In an embodiment, the electromagnetic energy 110 is then output or emitted from the electromagnetic energy source 108 (under the control of the control system 116) toward the target region 112 to heat the target region 112. The presence of the electromagnetic energy absorption agent 338 in the target region 112 can enhance absorption of the electromagnetic energy 110 to further heat the target region 112. In an embodiment, prior to, substantially and simultaneously with, or after the electromagnetic energy source 108 outputs the electromagnetic energy 110, the one or more sensors 114 determine the temperature of the target region 112. Thus, the electromagnetic energy absorption agent 338 enhances heating of the target region 112.

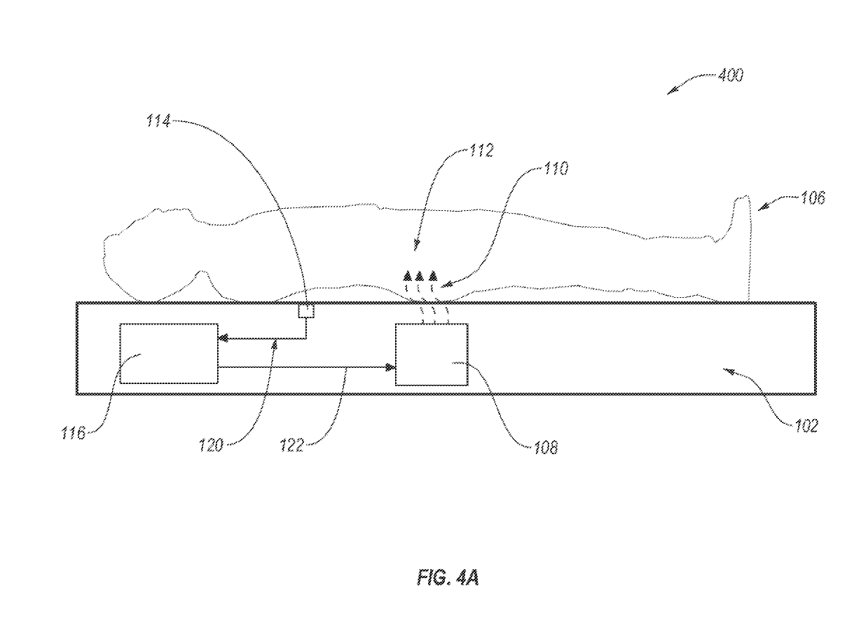

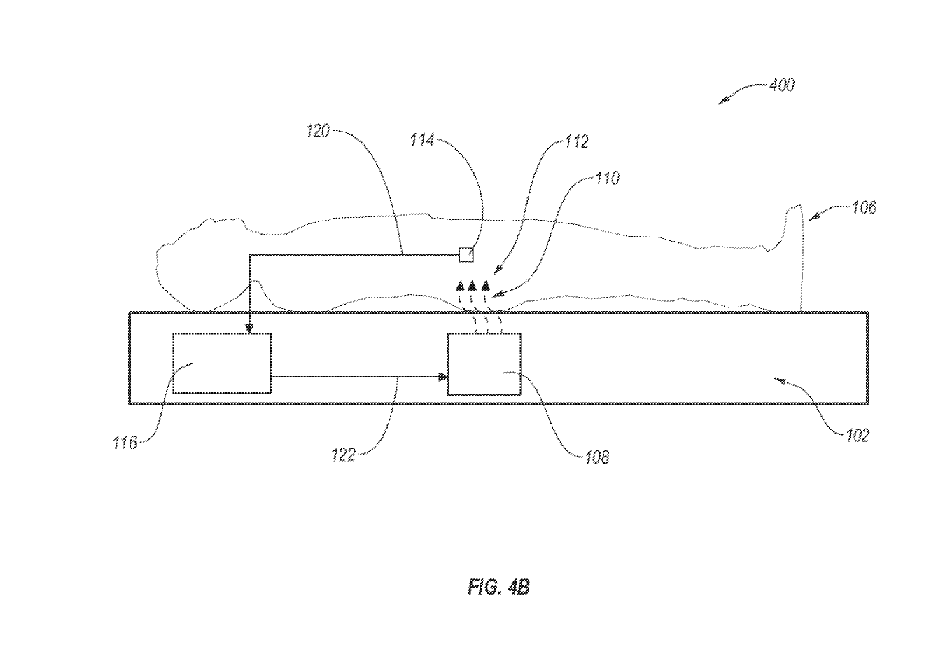

FIG. 4A is a schematic diagram of an embodiment of a hypothermia treatment system 400 configured as a self-contained unit that includes all functionalities necessary for the operation of the hypothermia treatment system 400. The hypothermia treatment system 400 includes many of the same components as the hypothermia treatment systems 100 and 300 shown in FIGS. 1A through 3. Therefore, in the interest of brevity, components of the hypothermia treatment system 400 that are identical or similar to each other have been provided with the same reference numerals, and an explanation of their structure and function will not be repeated unless the components function differently in the hypothermia treatment systems 100, 300, and 400. However, it should be noted that the principles of the hypothermia treatment system 400 are employed with any of the embodiments described with respect to FIGS. 1A through 3.

The hypothermia treatment system 400 can include a support structure 102 configured to support a subject 106. The hypothermia treatment system 400 further includes an electromagnetic energy source 108 configured to selectively output electromagnetic energy 110 toward a target region 112 of the subject 106 to heat the target region 112. Heating the target region 112 with the electromagnetic energy 110 can increase the core body temperature of the subject 106 via thermal conduction, thermal convection, or thermal radiation. In the illustrated embodiment, the electromagnetic energy source 108 is incorporated in the support structure 102. The hypothermia treatment system 400 can further include one or more sensors 114 that are also incorporated in the support structure 102. The one or more sensors 114 are configured to determine a temperature of the target region 112 of the subject 106. The hypothermia treatment system 400 also includes a control system 116 operably coupled to the electromagnetic energy source 108 and the one or more sensors 114. In the illustrated embodiment, the control system 116 is also incorporated in the support structure 102. Thus, the support structure 102, the electromagnetic energy source 108, and the one or more sensors 114 can form a single unit including all functionalities necessary for the operation of the hypothermia treatment system 400.

Referring to FIG. 4B, in an embodiment, the one or more sensors 114 are positioned internally within the subject 106. In an embodiment, the one or more sensors 114 are delivered to the subject 106 orally, topically, via injection, via implantation, or another suitable delivery method. In an embodiment, the one or more sensors 114 are delivered to the target region 112 within the subject 106. In an embodiment, the one or more sensors 114 can include a temperature probe positioned within the thorax of the subject 106. For example, the one or more sensors 114 can include a temperature probe delivered to the thorax via a catheter, implantation, or inhalation. In an embodiment, the one or more sensors 114 include a chip sensor or biosensors positioned within the arteries of the subject 106. For example, the one or more sensors 114 can be delivered intra-arterial via a catheter. In an embodiment, the one or more sensors 114 are positioned within rectum of the subject 106. For example, the one or more sensors 114 can be delivered to the rectum via a suppository or enema. In an embodiment, the one or more sensors 114 are positioned within the subcutaneous tissue of the subject 106. For example, in an embodiment, the one or more sensors 114 can be implanted in the subcutaneous tissue of the subject 106. In an embodiment, the one or more sensors 114 are delivered to the subject 106 intramuscularly. In an embodiment, the one or more sensors 114 can travel within the subject 106. For example, the one or more sensors 114 are delivered to one or more arteries or blood vessels of the subject 106 and configured to travel via blood flow. In an embodiment, the one or more sensors 114 are configured to travel via the gastrointestinal tract. In an embodiment, the control system 116 is wirelessly connected to the one or more sensors 114.

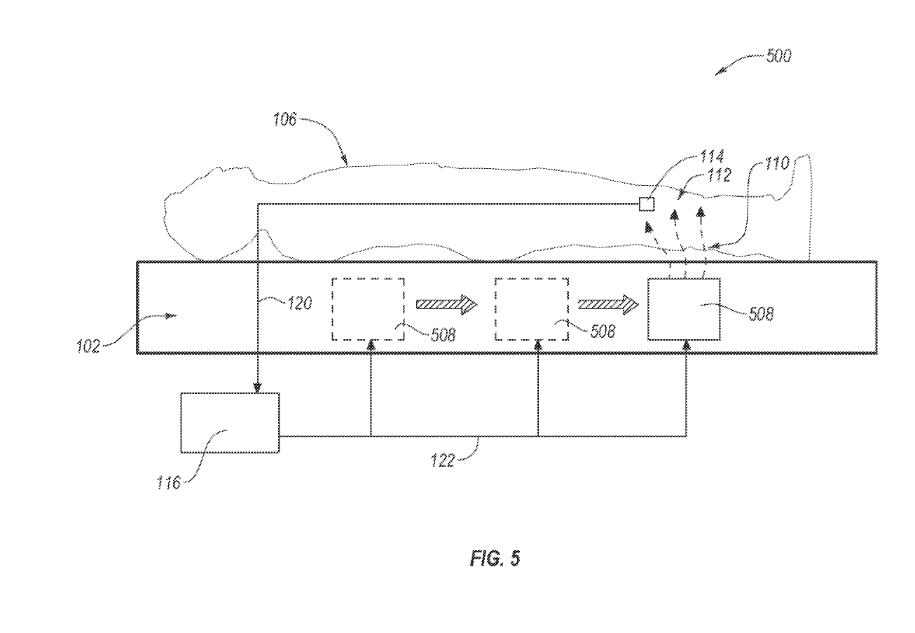

FIG. 5 is a schematic diagram of an embodiment of a hypothermia treatment system 500 including a movable electromagnetic energy source 508. The hypothermia treatment system 500 includes many of the same components as the hypothermia treatment systems 100, 300, and 400 shown in FIGS. 1A through 4B. Therefore, in the interest of brevity, components of the hypothermia treatment system 500 that are identical or similar to each other have been provided with the same reference numerals, and an explanation of their structure and function will not be repeated unless the components function differently in the hypothermia treatment systems 100, 300, 400, and 500. However, it should be noted that the principles of the hypothermia treatment system 500 are employed with any of the embodiments described with respect to FIGS. 1A through 4B.

The hypothermia treatment system 500 includes a support structure 102 configured to support a subject 106 exhibiting or in risk of exhibiting symptoms of hypothermia. An electromagnetic energy source 508 is incorporated in the support structure 102. The electromagnetic energy source 508 is configured to selectively output electromagnetic energy 110 toward a target region 112 of the subject 106 to heat the target region 112. Heating the target region 112 with the electromagnetic energy 110 can increase the core body temperature of the subject 106 via thermal conduction, thermal convection, or thermal radiation. In an embodiment, the target region 112 can include one or more locations on or within the body of the subject 106. The electromagnetic energy source 508 can include, but is not limited to, a microwave energy source, a phase-array energy source, a metamaterial array energy source, a radio-frequency energy source, a light source, a laser, a semiconductor laser, or a magnetic energy source. The hypothermia treatment system 500 further includes one or more sensors 114 that are positioned within the subject 106. The one or more sensors 114 can be configured to determine a temperature or other characteristics of the target region 112 of the subject 106. The hypothermia treatment system 500 further includes a control system 116 including control electrical circuitry (not shown). The control system 116 is operably coupled to the electromagnetic energy source 508 and the one or more sensors 114 to control operation of one or more of the foregoing system components.

The electromagnetic energy source 508 is movable relative to the support structure 102 or the subject 106. The electromagnetic energy source 508 can be configured to rotate, articulate, and translate relative to the support structure 102 or the subject 106. In an embodiment, the electromagnetic energy source 508 is movable via a track system incorporated in the support structure 102. In an embodiment, the electromagnetic energy source 508 is movable via an articulating arm operably coupled to the electromagnetic energy source 508. In operation, the control system 116 outputs one or more emitting information signals 120 to the electromagnetic energy source 508. The one or more emitting information signals 120 encode emitting information or directions. Responsive to the emitting information signals 122, the electromagnetic energy source 508 can move, aim, or emit the electromagnetic source 508. In an embodiment, the one or more emitting information signals 122 include or more directions to aim or direct the electromagnetic energy source 508 at the target region 112. In an embodiment, the one or more emitting information signals 122 include or more directions to align the electromagnetic energy source 508 with the target region 112. In an embodiment, the one or more emitting information signals 122 include one or more directions to move the electromagnetic energy source 508 toward the target region 112. In an embodiment, the one or more emitting information signals 122 include one or more directions to move the electromagnetic energy source 508 away from the target region 112. In an embodiment, the one or more emitting information signals 122 include one or more directions to emit the electromagnetic energy 110. In an embodiment, the one or more emitting information signals 122 include one or more directions to stop emitting the electromagnetic energy 110. Thus, in an embodiment, the control system 116 is configured to control movement and operation of the electromagnetic energy source 508.

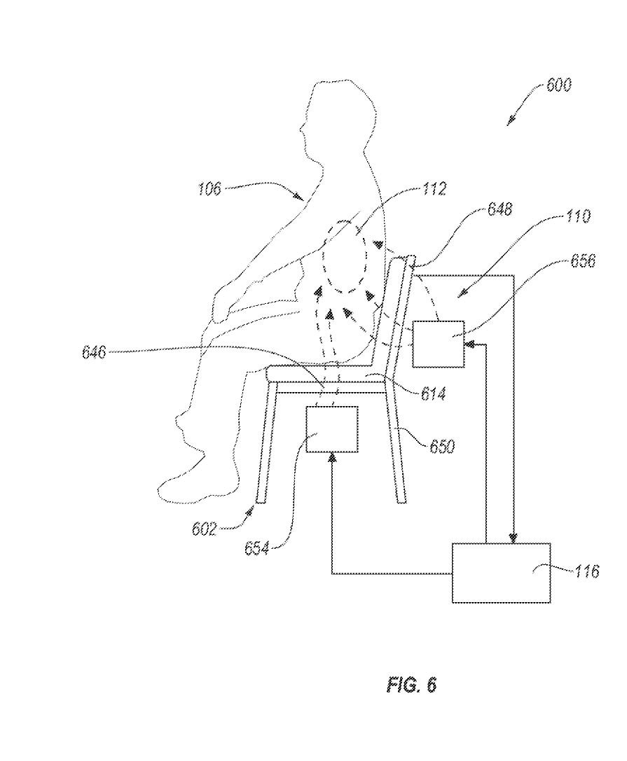

FIG. 6 is a schematic diagram of an embodiment of a hypothermia treatment system 600 including a support structure including a chair. The hypothermia treatment system 600 includes many of the same components as the hypothermia treatment systems 100, 300, 400, and 500 shown in FIGS. 1A through 5. Therefore, in the interest of brevity, components of the hypothermia treatment system 600 that are identical or similar to each other have been provided with the same reference numerals, and an explanation of their structure and function will not be repeated unless the components function differently in the hypothermia treatment systems 100, 300, 400, 500, and 600. However, it should be noted that the principles of the hypothermia treatment system 600 are employed with any of the embodiments described with respect to FIGS. 1A through 5.

The hypothermia treatment system 600 includes a support structure 602. As described above, the support structure 602 can exhibit a variety of different configurations selected for a particular application. For example, the support structure 602 can include a bed, a surgical table, a stretcher, a gurney, a couch, a sleeping bag, or a hypothermia wrap. In the illustrated embodiment, the support structure 602 includes a chair having a seat portion 646 and a back portion 648 extending upward from the seat portion 646. The chair 602 further includes a plurality of legs 650 extending downward from the seat portion 646. Thus, the subject 106 can be positioned in the chair 602 in a seating position.

A first electromagnetic energy source 654 is positioned under the seat portion 646 of the chair 602. A second electromagnetic energy source 656 is positioned on the back portion 650 of the chair 602. The first electromagnetic energy source 654 and the second electromagnetic energy source 656 are configured to selectively output electromagnetic energy 110 toward a target region 112 of the subject 106 to heat the target region 112. In an embodiment, the target region 112 can include one or more locations on or within the body of the subject 106. The first electromagnetic energy source 654 and the second electromagnetic energy source 656 can include, but is not limited to, a microwave energy source, a phase-array energy source, a radio-frequency energy source, or a magnetic energy source. In an embodiment, the first electromagnetic energy source 654 and the second electromagnetic energy source 656 are the same. In an embodiment, the first electromagnetic energy source 654 and the second electromagnetic energy source 656 are different from one another. The hypothermia treatment system 600 further includes one or more sensors 614 that are positionable on the seat portion 646 and the back portion 648 of the chair. In an embodiment, the one or more sensors 614 can include a sheet positioned between the chair 602 and the subject 106. The one or more sensors 614 can be configured to determine a temperature or other characteristics of the target region 112 of the subject 106. The hypothermia treatment system 600 further includes a control system 116 including control electrical circuitry (not shown). The control system 116 is operably coupled to the first electromagnetic energy source 654, the second electromagnetic energy source 656, and the one or more sensors 614 to control operation of one or more of the foregoing system components.

It will be understood that a wide range of hardware, software, firmware, or virtually any combination thereof can be used in the controllers described herein. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more programs running on one or more processors (e.g., as one or more programs running on one or more microprocessors), as firmware, or as virtually any combination thereof. In addition, the reader will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, the various embodiments described herein can be implemented, individually and/or collectively, by various types of electro-mechanical systems having a wide range of electrical components such as hardware, software, firmware, or virtually any combination thereof and a wide range of components that can impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs.

In a general sense, the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein can be implemented in an analog or digital fashion or some combination thereof.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

In some instances, one or more components can be referred to herein as “configured to.” The reader will recognize that “configured to” or “adapted to” are synonymous and can generally encompass active-state components and/or inactive-state components and/or standby-state components, unless context requires otherwise.

While particular aspects of the present subject matter described herein have been shown and described, it will be apparent that, based upon the teachings herein, changes and modifications can be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, any recited operations therein can generally be performed in any order. Examples of such alternate orderings can include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, the various aspects and embodiments disclosed herein are for purposes of illustration and are not intended to be limiting, with the true scope and spirit being indicated by the following claims.

What is claimed is:

1. A system for treating hypothermia in a subject, the system comprising:
a support structure configured to support the subject thereon, the subject having a target region;
an electromagnetic energy source configured to output electromagnetic energy towards the target region of the subject to selectively heat the target region, the electromagnetic energy source located external to the subject;
at least one temperature sensor configured to determine or measure a temperature of the target region of the subject; and
a control system operably coupled to the electromagnetic energy source and the at least one temperature sensor, the control system configured to control at least one operational parameter of the electromagnetic energy output by the electromagnetic energy source responsive to the temperature sensor determining or measuring the temperature of the target region so that the temperature of the target region is maintained below a tissue damaging temperature of the target region.

2. (canceled)

3. The system of claim 1, wherein the at least one temperature sensor is included in a thermography system.

4. The system of claim 3, wherein the thermography system includes at least one of a magnetic resonance imaging system or a radiography system.

5. The system of claim 3, wherein the thermography system includes an electromagnetic energy source antenna, and wherein the at least one temperature sensor shares at least one component with the electromagnetic energy source antenna.

6. The system of claim 1, wherein the at least one temperature sensor is configured to be deployed internally within the subject.

7. The system of claim 1, wherein the at least one temperature sensor is configured to be deployed external to the subject.

8. The system of claim 1, wherein the at least one temperature sensor measures skin temperature.

9. The system of claim 1, wherein the control system is configured to determine an electromagnetic energy irradiation profile for the target region at least partially based on physiological data of the subject.

10. (canceled)

11. (canceled)

12. The system of claim 1, wherein the support structure includes a reflective material that is reflective to the electromagnetic energy.

13. The system of claim 1, further including a patient covering including a reflective material.

14. (canceled)

15. The system of claim 1, wherein the support structure includes an electromagnetic energy absorptive material disposed thereon that is absorptive to the electromagnetic energy.

16. The system of claim 1, further including a patient covering including an energy absorptive material.

17. The system of claim 1, wherein the electromagnetic energy source includes a microwave energy source, and wherein the electromagnetic energy includes microwave energy.

18. The system of claim 1, wherein the electromagnetic energy source includes a steerable microwave energy source, and wherein the electromagnetic energy includes microwave energy.

19. (canceled)

20. (canceled)

21. The system of claim 1, wherein the electromagnetic energy source includes a radio-frequency energy source, and wherein the electromagnetic energy includes radio-frequency energy.

22. The system of claim 1, wherein the electromagnetic energy source includes a magnetic energy source, and wherein the electromagnetic energy includes magnetic energy.

23. The system of claim 1, wherein the at least one operational parameter of the electromagnetic energy includes at least one of location of the electromagnetic energy, direction of the electromagnetic energy, intensity of the electromagnetic energy, duration of the electromagnetic energy applied to the target region, frequency of the electromagnetic energy, phase of the electromagnetic energy, or pulse frequency of the electromagnetic energy.

24. The system of claim 1, wherein the control system is configured to control the at least one operational parameter to vary an electromagnetic absorption profile within the subject.

25. The system of claim 1, wherein the control system is configured to control the at least one operational parameter to vary the direction of the electromagnetic energy.

26. The system of claim 1, wherein the control system is configured to control the at least one operational parameter so that the temperature of the target region is maintained below about 40° C.

27. The system of claim 1, wherein the control system is configured to control the at least one operational parameter so that the temperature of the target region is substantially uniform therewithin.

28. The system of claim 1, wherein the electromagnetic energy source is positioned underneath the support structure.

29. The system of claim 1, wherein the electromagnetic energy source is incorporated in the support structure.

30. (canceled)

31. The system of claim 1, wherein the control system includes a processor that numerically models at least one of electromagnetic propagation, energy absorption, or thermal transport.

32. The system of claim 1, wherein the control system includes a processor that numerically models thermal transport with the patient resulting from absorption of the electromagnetic energy.

33. The system of claim 1, further including:
a supply of electromagnetic energy absorption agent having a peak absorption temperature below about 40° C.;
a delivery device configured to inject the electromagnetic energy absorption agent from the supply of electromagnetic energy absorption agent internally into the subject; and
wherein the control system controls the at least one at least one operational parameter of the electromagnetic energy to maximize absorption by the electromagnetic energy absorption agent.

34. A method for treating hypothermia in a subject, the method comprising:
determining a temperature indicative of the temperature of a subsurface target region of the subject; and
responsive to determining the temperature, directing electromagnetic energy at an external surface of the target region of the subject effective to heat the subsurface target region to a temperature of less than a tissue damaging temperature of the subsurface target region.

35. The method of claim 34, wherein the tissue damaging temperature is less than about 40° C.

36. The method of claim 34, further including controlling at least one operational parameter of the electromagnetic energy source so that the temperature of the target region is maintained below about 40° C.

37. The method of claim 36, wherein the controlling the at least one operational parameter includes controlling at least one of a location of the electromagnetic energy, a direction of the electromagnetic energy, an intensity of the electromagnetic energy, duration of the electromagnetic energy applied to the subsurface target region, frequency of the electromagnetic energy, phase of the electromagnetic energy, or a pulse frequency of the electromagnetic energy.

38. The method of claim 34, further including controlling at least one operational parameter of the electromagnetic energy responsive to measuring the temperature so that the temperature of the subsurface target region is substantially uniform.

39. The method of claim 34, further including determining an electromagnetic energy irradiation profile for the subsurface target region at least partially based on physiological data of the subject.

40. The method of claim 39, further including receiving the physiological data from a medical imaging instrument.

41. (canceled)

42. The method of claim 34, further including supporting the subject on a support structure having a reflective material thereon that is reflective to the electromagnetic energy.

43. The method of claim 34, further including supporting the subject on a support structure having an energy absorptive material thereon that is absorptive to the electromagnetic energy.

44. The method of claim 34, further including at least partially covering the subject with a material that is reflective to the electromagnetic energy.

45. The method of claim 34, further including at least partially covering the subject with a material that is absorptive to the electromagnetic energy.

46. The method of claim 34, wherein the electromagnetic energy includes at least one of radio-frequency energy, microwave energy, or magnetic energy.

47. (canceled)

48. (canceled)

49. (canceled)

50. The method of claim 34, wherein the temperature indicative of the temperature of the subsurface target region is determined substantially simultaneously with directing the electromagnetic energy at the external surface of the target region.

51. (canceled)

52. The method of claim 49, further including operating a temperature sensor during a pause in the operation of the electromagnetic energy source.

53. The method of claim 34, further including determining an electromagnetic absorption pattern for the target region at least partially based on the physiological data of the subject.

54. (canceled)

55. (canceled)

56. (canceled)

57. The method of claim 34, further including numerically modeling at least one of the electromagnetic propagation, energy absorption, or thermal transport.

58. (canceled)

59. (canceled)

60. The method of claim 34, further including:
introducing an electromagnetic energy absorption agent internally into the subject; and
wherein directing the electromagnetic energy at the external surface of the target region of the subject includes directing the electromagnetic energy at the electromagnetic energy absorption agent.

61. The method of claim 60, wherein introducing an electromagnetic energy absorption agent includes introducing the electromagnetic energy absorption agent into a blood stream of the subject.

62. (canceled)

63. (canceled)

64. The method of claim 34, wherein determining a temperature indicative of the temperature of a subsurface target region of the subject includes measuring the temperature with an external temperature sensor.

65. The method of claim 34, wherein determining a temperature indicative of the temperature of a subsurface target region of the subject includes measuring the temperature with a temperature sensor disposed internally within the subject.
